(19) United States
(12) Patent Application Publication
(10) Pub. No.: US 2021/0125718 A1
(43) Pub. Date: Apr. 29, 2021

(54) PROGRAM, APPARATUS, SYSTEM AND METHOD FOR HEALTH-RELATED INFORMATION MANAGEMENT

(71) Applicant: CureApp, Inc., Tokyo (JP)

(72) Inventor: Shin Suzuki, Chuo-ku, Tokyo (JP)

(73) Assignee: CureApp, Inc., Tokyo (JP)

(21) Appl. No.: 16/647,205

(22) PCT Filed: Sep. 18, 2018
(86) PCT No.: PCT/JP2018/034446

§ 371 Date: Sep. 1, 2020

(30) Foreign Application Priority Data
Sep. 15, 2017 (JP) PCT/JP2017/033557

(51) Int. Cl. G16H 40/67; G16H 20/30; A61B 5/11

(52) U.S. Cl. CPC G16H 40/67; A61B 5/1118; G16H 20/30

(57) ABSTRACT

A health-related information management system includes one or more electronic devices. At least one of the one or more electronic devices: measures measurement data including a measurement value, and a measurement timing of the measurement value; stores the measurement data and the measurement timing; determines, using the measurement timing and a timing to present a measurement result using the measurement data, whether the measurement data satisfies a pre-set presentation timing condition; and presents, as the measurement result, only measurement data among the measurement data that satisfy the pre-set presentation timing condition.

### TECHNICAL FIELD

The present invention relates to a program, device, system and method for health-related information management.

### BACKGROUND

In health management, it is important to measure biological information such as body weight and body fat, and information related to health (health-related information) including physical activity information such as the number of steps, and improve the daily life so as to allow the resulting measurement value to become a desirable value. A measurement value of body weight, body fat or the like is likely to vary depending on living conditions of a day, so that it is difficult to accurately figure out a true or real change in body weight or the like. Therefore, there has been proposed a system configured to measure body weight or body fat, and display the resulting measurement value and a past measurement value in the form of a graph, thereby making it possible to accurately figure out a change in body weight or the like, as a long-term transition (Document 1).

### RELATED ART DOCUMENTS

Document 1: JP 2001-190514A

However, most persons who need to improve their lifestyle are more likely to be sensitive to a measurement value of body weight or body fat of each day, particularly a measurement value of a day when it was measured, rather than a long-term transition thereof. Although a measurement value of body weight or the like is likely to vary depending on living conditions of a day, many persons react nervously to rise/fall of a measurement value of body weight or the like of each day. Specifically, there are many cases where a person is largely discouraged by a measurement value of body weight of the day when he/she occasionally ate a lot, and stops the measurement after that, finally leading to abandonment of an effort to improve his/her health state. On the other hand, there is also a case where, when a measurement value of body weight exhibits a significant decease because a person occasionally makes the measurement after exercise, the person will neglect to improve his/her habitus.

Further, it is effective for improvement in health state to carry out physical activity such as sustained exercise, and, for example, walking is known as one effective exercise. The number of steps can be measured by using a pedometer. Although it is important for health promotion or the like to continuously walk every day by a given number or more of steps, a person unnecessarily gets satisfaction from a measurement value measured by a pedometer on the day when he/she occasionally walked by a relatively large number of steps, and thereby will neglect walking, from the next day, in some cases. On the other hand, there are also many cases where a person is largely discouraged by a small number of steps measured by a pedometer on the day when he/she occasionally walked by a relatively small number of steps, and stops the measurement after that, finally leading to abandonment of an effort to improve his/her health state.

### SUMMARY

One or more embodiments of the present invention provide a program, device, system and method for health-related information management, each designed to adequately manage health-related information so as to improve health state.

According to one or more embodiments of the present invention, there is provided a health-related information management system for presenting a measurement result based on measurement data satisfying a pre-set presentation timing condition. The health-related information management system comprises one or more electronic devices, wherein: at least one of the one or more electronic devices that stores measurement data including measurement values measured by a health-related information measurement device, and information indicative of a measurement timing of the measurement values; at least one of the one or more electronic devices that determines measurement data satisfying the presentation timing condition based on the information indicative of the measurement timing and a timing to present the measurement result based on the measurement data; and at least one of the one or more electronic devices that presents the measurement result based on the measurement data determined to satisfy the presentation timing condition, but not present measurement results based on measurement data failing to satisfy the presentation timing condition.

In the health-related information management system according to one or more embodiments of the present invention, the information indicative of the measurement timing includes information indicative of a measurement time of the measurement value, and the presentation timing condition includes a predetermined day-of-week or day-of-month after the measurement time, or a timing after a given time period has elapsed from the measurement time.

In the health-related information management system according to one or more embodiments of the present invention, the information indicative of the measurement timing includes information indicative of a number of measurements performed after the measurement of the measurement value, and the presentation timing condition includes a timing after a given number or more of measurements have been performed since the measurement of the measurement value.

In the health-related information management system according to the first aspect of the present invention, when the measurement value satisfies a criterion based on a value indicative of a tendency of measurement values, the measurement value is determined to satisfy the presentation timing condition.

In the health-related information management system according to one or more embodiments the present invention, at least one of the one or more electronic devices that accepts an input of an index (an index input) made by a user, or a measured person, of the health-related information measurement device, wherein the index is indicative of a factor related to the health-related information of the user at the time of the measurement of the health-related information of the user, and is associated with the measurement value, and wherein the measurement data includes the index.

In the above health-related information management system, the health-related information is information about body weight, and wherein the index is indicative of at least one of a state after urination at the time of awakening, a state after eating, a state after taking a bath, a state after exercise, a dressed state, and a state during a menstrual period.

In the health-related information management system according to one or more embodiments of the present invention, the one or more electronic devices comprise a health-related information measurement device and a user-side device.

In the health-related information management system according to one or more embodiments of the present invention, at least one of the one or more electronic devices that receives an input of a prediction (a prediction input) made by a user of the health-related information measurement device, wherein the prediction is made as to whether the measurement value which is targeted for the prediction increases or decreases as compared with the previous measurement value, and wherein at least one of the one or more electronic devices that compares the target measurement value with the previous measurement value, to perform a correct/incorrect determination as to whether or not the prediction made by the user is correct, and wherein the measurement data includes a result of the correct/incorrect determination.

In the health-related information management system according to one or more embodiments of the present invention, the presenting the measurement result includes presenting a graph based on the measurement data which include the measurement values measured within a given time period.

In the health-related information management system according to one or more embodiments of the present invention, the one or more electronic devices comprise: a health-related information measurement device; a user-side device; and a server connected to the health-related information measurement device and the user-side device via a network.

According to one or more embodiments of the present invention, a user-side device for use in a health-related information management system for presenting a measurement result based on measurement data satisfying a pre-set presentation timing condition, wherein the user-side device that presents the measurement result based on the measurement data which are a part of measurement data including measurement values measured by a health-related information measurement device and information indicative of measurement timings of the measurement values, and are determined to satisfy the presentation timing condition, based on the information indicative of the measurement timing and a timing to present the measurement result based on the measurement data, but not to present measurement results based on measurement data failing to satisfy the presentation timing condition.

According to one or more embodiments of the present invention, there is provided a method performed in a health-related information management system for presenting a measurement result based on measurement data satisfying a pre-set presentation timing condition, wherein the health-related information management system comprises one or more electronic devices. The method comprises: storing, by at least one of the one or more electronic devices, measurement data including measurement values measured by a health-related information measurement device, and information indicative of measurement timings of the measurement values; determining, by at least one of the one or more electronic devices, the measurement data satisfying the presentation timing condition based on the information indicative of the measurement timing and a timing to present the measurement result based on the measurement data; and presenting, by at least one of the one or more electronic devices, the measurement result based on the measurement data determined to satisfy the presentation timing condition, but not presenting measurement results based on the measurement data failing to satisfy the presentation timing condition.

According to one or more embodiments of the present invention, there is provided a set of programs for causing one or more electronic devices to perform the above method.

According to one or more embodiments of the present invention, there is provided a method performed by a user-side device for use in a health-related information management system for presenting a measurement result based on a measurement data satisfying a pre-set presentation timing condition. The method comprises presenting the measurement result based on the measurement data which are a part of measurement data including measurement values measured by a health-related information measurement device and information indicative of measurement timings of the measurement values, and are determined to satisfy the presentation timing condition, based on the information indicative of the measurement timing and a timing to present the measurement results based on the measurement data, but not presenting measurement results based on measurement data failing to satisfy the presentation timing condition.

According to one or more embodiments of the present invention, there is provided a program for causing a user-side device to perform the above method.

One or more embodiments of the present invention makes it possible to perform health-related information management in such a manner as to present, to a user of the health-related information measurement device for measuring health-related information, the measurement results based on the measurement data satisfying the pre-set presentation timing condition, but not presenting, to the user, measurement results based on measurement data failing to satisfy the presentation timing condition. This makes it possible to prevent the user from reacting nervously to a measurement value of each day, and allow the user to sustainably perform the measurement and thus make an effort for sustained health improvement.

### BRIEF DESCRIPTION OF DRAWINGS

FIG. 1 is a configuration diagram of a system according to a first embodiment of the present invention.

FIG. 2 is a hardware configuration diagram of a health-related information measurement device in the system according to the first embodiment.

FIG. 3 is a hardware configuration diagram of a user-side device in the system according to the first embodiment.

FIG. 4 is a functional block diagram of the health-related information measurement device in the system according to the first embodiment.

FIG. 5 is a functional block diagram of the user-side device in the system according to the first embodiment.

FIG. 6 is a flowchart of the operation of the system according to the first embodiment.

FIGS. 7(a) to 7(d) are examples of a display image on the user-side device in the system according to the first embodiment.

FIG. 8 is a configuration diagram of a system according to a second embodiment of the present invention.

FIG. 9 is a hardware configuration diagram of a server device in the system according to the second embodiment.

FIG. 10 is a functional block diagram of the server device in the system according to the second embodiment.

FIG. 11 is a flowchart of the operation of the system according to the second embodiment.

FIG. 12 is a flowchart of the operation of a system according to a third embodiment of the present invention.

FIG. 13 is a flowchart of the operation of a system according to a fourth embodiment of the present invention.

### DETAILED DESCRIPTION

### First Embodiment

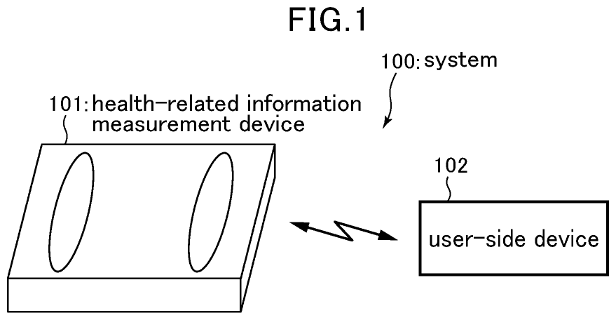

FIG. 1 is a configuration diagram of a system according to a first embodiment of the present invention. The system 100 is designed to be used for managing health-related information, and comprises a health-related information measurement device 101, and a user-side device 102 to be used by a user of the health-related information measurement device 101. The health-related information measurement device 101 and the user-side device 102 are connected via wire communication or wireless communication.

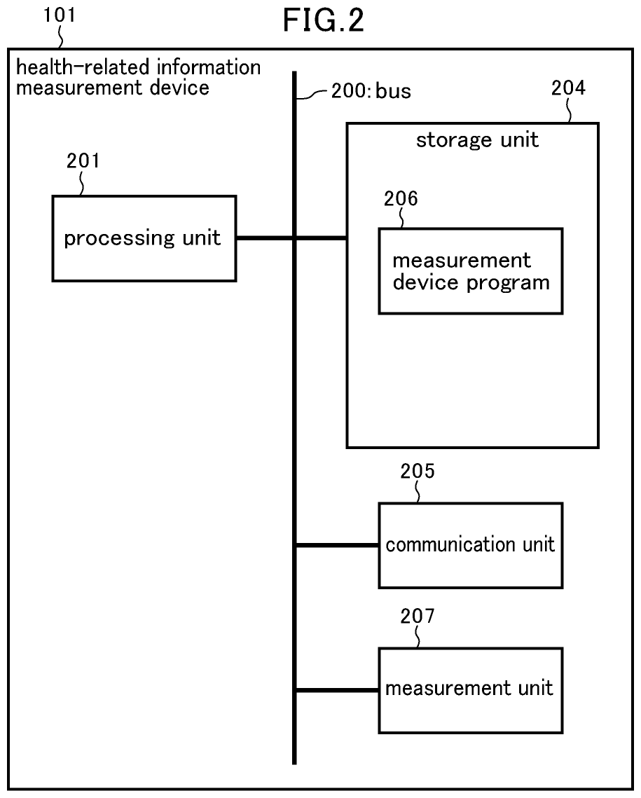

FIG. 2 is a hardware configuration diagram of the health-related information measurement device 102 in the system 100 according to the first embodiment. The health-related information measurement device 101 is an electronic device which comprises a processing unit 201, a storage unit 204, a communication unit 205, and a measurement unit 207. These units are connected to each other via a bus 200. However, the system may be configured such that two or more of the units are connected together, on an as-needed or case-by-case basis. Although this embodiment will be described based on an example where the health-related information measurement device 101 is composed of a body weight scale (bathroom scale), the health-related information measurement device may further comprise a body fat scale for measuring a biological impedance of the user, or may be a device for measuring any other health-related information including biological information such as blood pressure or heart rate, and physical activity-related information such as the number of steps. As used in this specification, the term “measurement” includes scaling, gauging or the like, and the health-related information measurement device encompasses any device capable of measuring (scaling or gauging) information related to health.

In this embodiment, the health-related information measurement device 101 is devoid of a display unit 202 and an input unit 203. However, it should be understood that the health-related information measurement device 101 may be equipped with them. The storage unit 204 stores therein a health-related information measurement device program 206. The storage unit 204 may be any type of storage unit such as a non-volatile memory or a volatile memory, as long as it is capable of storing information therein. The communication unit 205 that performs wire communication using an Ethernet (trademark) cable or the like, or wireless communication using Bluetooth (trademark) connection, wireless LAN or the like, to establish connection to the user-side device 102.

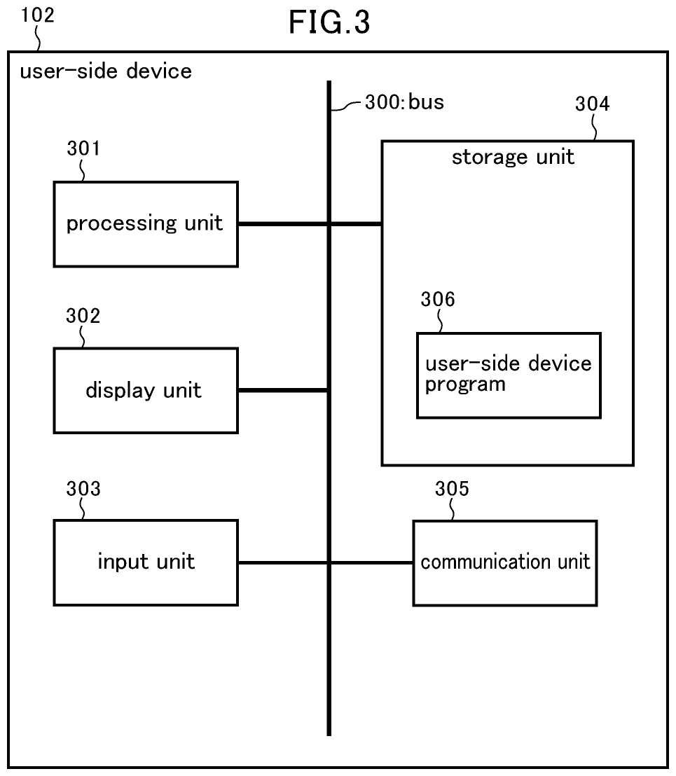

FIG. 3 is a hardware configuration diagram of the user-side device 102 which is an electronic device to be used by the user. The user-side device 102 comprises a processing unit 301, a display unit 302, an input unit 303, a storage unit 304, and a communication unit 305. These units are connected to each other via a bus 300. However, the system may be configured such that two or more of the units are connected together, on an as-needed or case-by-case basis. The user-side device 102 may be composed of a desktop computer or a notebook computer, or may be a personal digital assistance, a mobile phone, a smartphone or a tablet terminal. The display unit 302 has a function of displaying information to the user. The input unit 303 has a function of receiving an input from the user, like a keyboard, a mouse or the like. In a case where the user-side device 102 is composed of a smartphone or a tablet terminal, the display unit 302 and the input unit 303 may be integrated together as a touch panel. The storage unit 304 stores therein a user-side device program 306 for the user-side device. The storage unit 304 may be any type of storage unit such as a non-volatile memory or a volatile memory, as long as it is capable of storing information therein.

The communication unit 305 that performs wire communication using an Ethernet (trademark) cable or the like, or wireless communication such as mobile communication, Bluetooth (trademark) communication or wireless LAN communication, to establish connection to the health-related information measurement device 101.

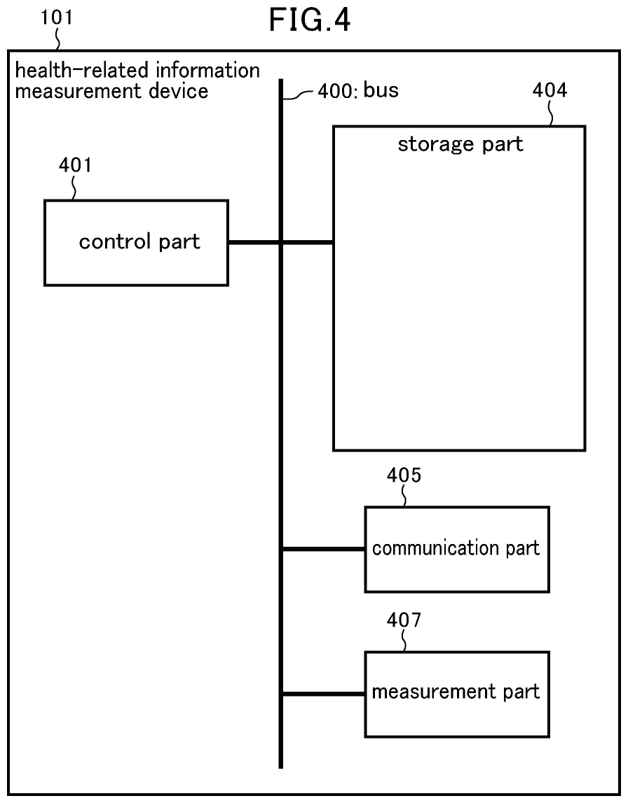

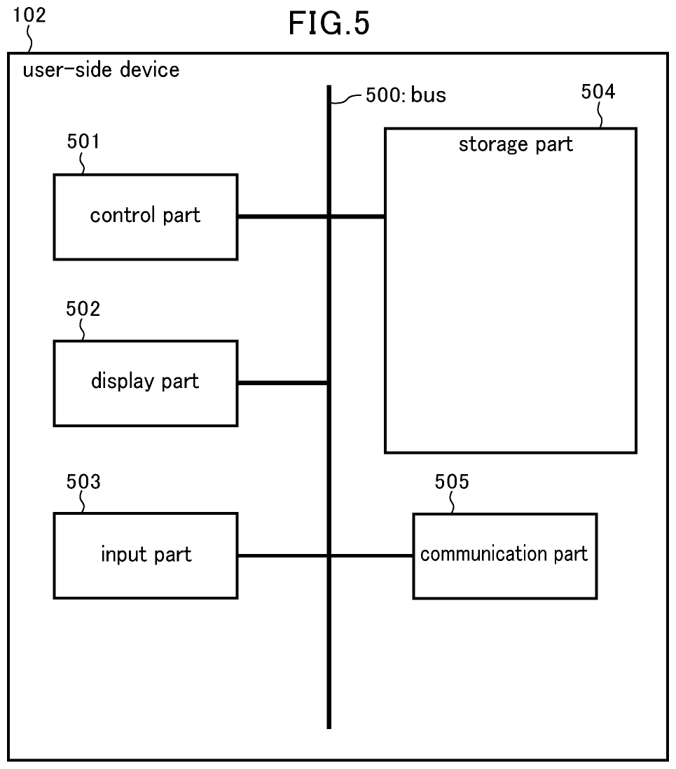

FIGS. 4 and 5 are, respectively, functional block diagrams of the health-related information measurement device 101 and the user-side device 102 in the system according to the first embodiment. The health-related information measurement device 101 comprises a control part 401, a storage part 404, a communication part 405, and a health-related information measurement part 407, and the user-side device 102 comprises a control part 501, a display part 502, an input part 503, a storage part 504, and a communication part 505. Each of the control parts 401, 501 has a function of executing controls such as information processing. The display part 502 has a function of allowing the user to visually view information. The input part 503 has a function of accepting an input from the user. Each of the storage parts 404, 504 has a function of storing therein data and others. Each of the communication parts 405, 505 has a function of performing transmitting and receiving of information with respect to another device. The health-related information measurement part 407 is operable to measure the health-related information of the user, and output the resulting measurement value. In this embodiment, the above functions are realized by executing each of the programs 206, 306 in a corresponding one of the processing units 201, 301 illustrated in FIGS. 2 and 3 to cause the hardware to co-operate with one another. Alternatively, these functions may be realized based on hardware by providing one or more electronic circuits or the like capable of realizing the functions.

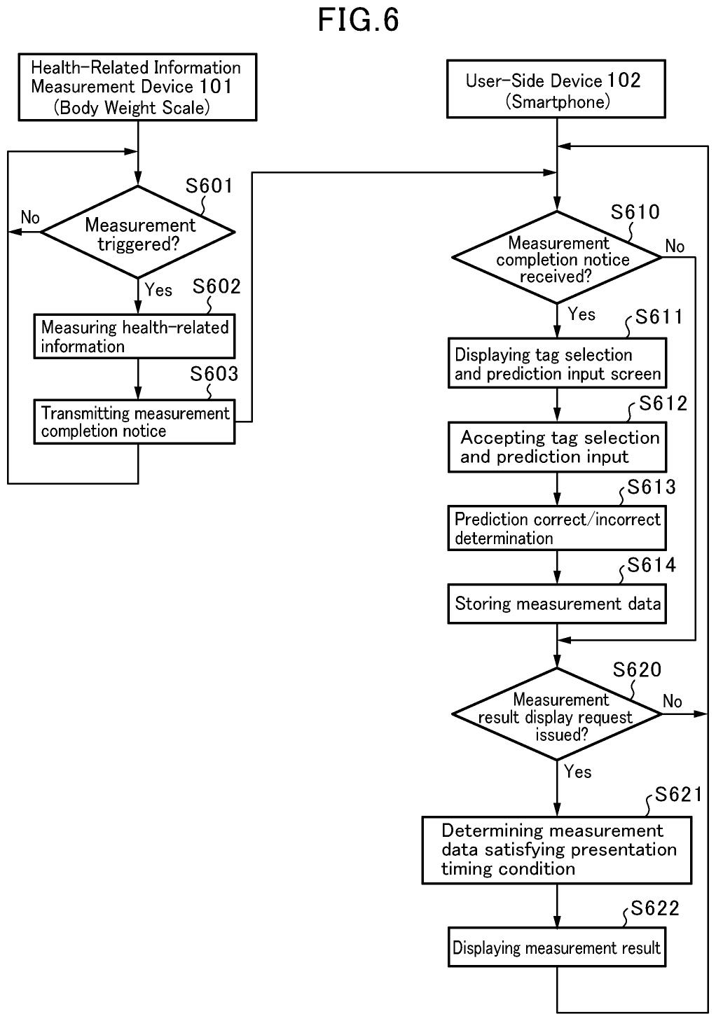

With reference to FIG. 6, the operation of the system according to this embodiment will be described. In this embodiment, a bath scale is used as the health-related information measurement device 101, and a smartphone is used as the user-side device 102. The health-related information measurement device 101 and the user-side device 102 are wirelessly connected to each other via a Bluetooth (trademark) connection. A health-related information measurement system-operating application for operating the system according to this embodiment is installed in the smartphone 102.

First of all, the control part 501 of the smartphone 102 operates to set, based on an input made by the user through the input part 503, a presentation timing condition for presenting a result of measurement of body weight to the user. Then, the control part 501 operates to determine measurement data satisfying the presentation timing condition, based on information indicative of a measurement timing when the measurement of body weight was performed, and a timing to present the measurement result based on the measurement data, and then present, to the user, the measurement result based on the measurement data determined to satisfy the presentation timing condition. In this embodiment, any one day-of-week of Sunday to Saturday is used as the presentation timing condition. Whether or not the presentation timing condition is satisfied is determined with respect to each measurement data including the measurement value, and a certain measurement data including a measurement time can satisfy the presentation timing condition, only on or after a day-of-week (used as the presentation timing condition) in a week next to a week including the measurement time. In this embodiment, the information indicative of the measurement timing is a time when the measurement was performed, as described later. However, the information indicative of the measurement timing may be any other information as long as it is indicative of a timing when the measurement was performed

For example, in a case where the presentation timing condition is set to Sunday, a measurement result based on measurement data measured before previous Sunday at 12:00 a.m., is presented, whereas a measurement result based on measurement data measured in a period from the previous Sunday at 12:00 a.m., until before the subsequent Sunday at 12:00 a.m., is not presented. After the next Sunday at 12:00 a.m., a measurement result based on measurement data measured before then is allowed to be presented, but a measurement result based on measurement data measured since then is not presented until the Sunday after the subsequent Sunday comes.

Although, in this embodiment, day-of-week is used as the presentation timing condition, the user-side device 102 may be configured to allow the user to set a time (clock time), in addition to day-of-week. Further, the elapse of a required time period may also be used as the presentation timing condition. In this case, the user-side device 102 may be configured to allow the measurement result based on the measurement value to be presented, when a required time period set to start from the measurement time has elapsed. Instead of day-of-week, date-and-time may be used as the presentation timing condition. Alternatively, the presentation timing condition may be set such that it is satisfied when, after completion of the measurement, a given number or more of further measurements have been performed. Alternatively, the presentation timing condition may be set such that it is satisfied when the measurement value satisfies a criterion based on a value indicative of a tendency of past measurement values. Further, the presentation timing condition may be any condition as long as it determines a timing for presenting the measurement result.

Next, a body weight measurement operation (as a health-related information measurement operation) by the body weight scale 101 and an information processing operation by the smartphone 102 will be described based on FIG. 6. Firstly, in a case where the user measures his/her body weight, upon power-on of the body weight scale 101, a Bluetooth (trademark) connection to the smartphone 102 is established. Then, the body weight scale 101 waits for the user to step thereon to start a measurement of body weight, and starts the measurement when the user steps thereon (S601). The control part 401 of the body weight scale 101 operates to instruct the measurement part 407 to measure the body weight of the user (S602), and, upon completion of the measurement, transmit a measurement completion notice containing a measurement value and a measurement time, to the smartphone 102 through the communication part 405 and via the Bluetooth connection (S603). The measurement completion notice needs not contain any information for explicitly notifying the user-side device 102 of the completion of the measurement. That is, it may be any notice as long as it can cause the user-side device 102 to display a tag input screen for the completed measurement. Further, the measurement completion notice may contain only a measurement value and a measurement time. After transmitting the measurement completion notice, the body weight scale 101 returns to a standby mode to wait for the next measurement directive.

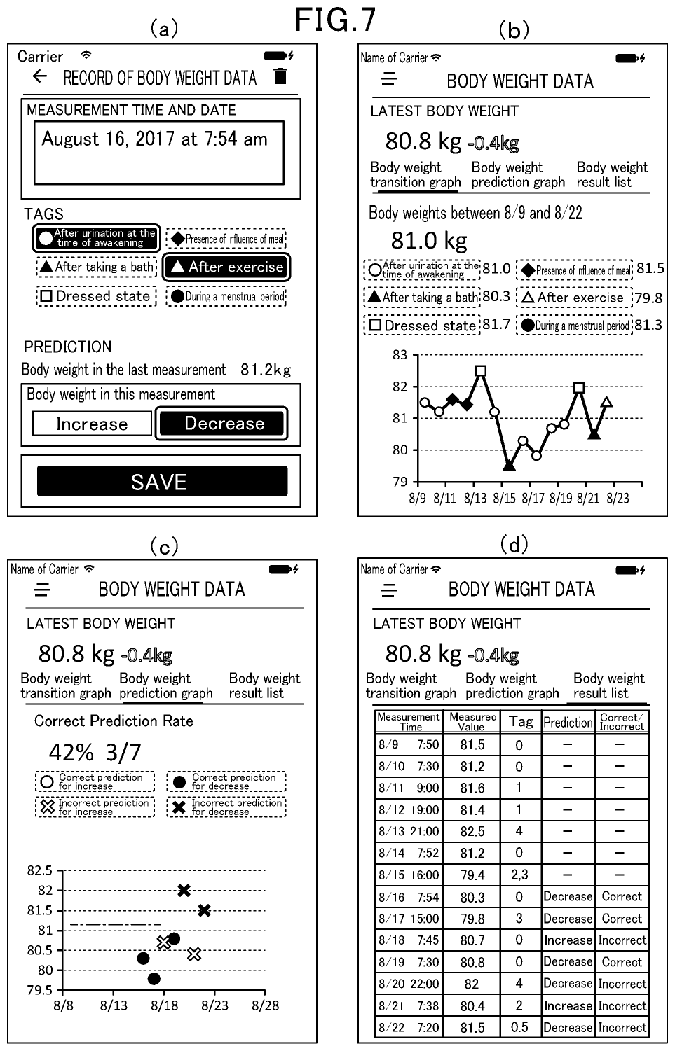

The control part 501 of the smartphone 102 operates to wait for a measurement completion notice (S610), and, upon receiving the measurement completion notice transmitted from the body weight scale 101 and containing the measurement value, display a message indicating the measurement time on the display part 502, thereby notifying the user of the fact that the measurement has been completed, and an input screen for prompting the user to input an index indicative of a factor related to biological information of the user at the time of the measurement, and his/her prediction as to whether the measurement value measured by the measurement increases or decreases as compared with a previous measurement value, as shown in FIG. 7(a) (S611). In this process, the result of this measurement is not displayed. The measurement result is presented only when it satisfies the presentation timing condition later. By keeping the measurement result from being presented just after the measurement, it become possible to prevent the user from reacting nervously to rise/fall of a measurement value of each day and thereby losing his/her motivation for improvement toward appropriate habitus. The measurement completion notice may be configured such that it does not contain any measurement time. In this case, a time when the smartphone 102 receives the measurement completion notice may be used as a measurement time.

In this embodiment, the input of an index indicative of a factor related to the health-related information of the user at the time of the measurement of the health-related information is realized by assigning a tag indicative of a factor which is considered to exert an influence on measurement value of body weight. The assignment of the tag, i.e., tagging, can be performed through selection from among a plurality of tags displayed on the input screen (FIG. 7(a)). The input screen may be configured to allow the user to assign more than one tag to a single measurement value. In this embodiment, among a state after urination at the time of awakening, a state in which there is an influence of meal, a state after taking a bath, a state after exercise a dressed state, and a state during a menstrual period, the user selects one or more of the tags corresponding to a state at the time of the measurement. A measurement value is considered to become most stable after urination at the time of awakening. On the other hand, a measurement value tends to become reduced after taking a bath or after exercise, and tends to become increased when eats a lot, or in a dressed state, or during a menstrual period. The tag is not limited to these, but may be a daily event, a specific context, or any other factor which is considered to exert an influence of the health-related information which is targeted for measurement, i.e., a measurement value of body weight here. A free-form input text with respect to each measurement is also one form of the index indicative of a factor related to the health-related information.

With regard to the prediction, the user inputs his/her prediction as to whether the measurement value obtained by this measurement increases or decreases as compared with a previous measurement value. As shown in FIG. 7(a), the previous measurement value may be displayed for the prediction. In a case where a measurement value can be checked immediately after the measurement, when a sufficient achievement cannot be obtained for effort, the user is likely to be discouraged, or is likely to be triggered to maintain interest in body weight and have a positive thought “I have to put more effort”. In the present invention, the user cannot immediately check a measurement value. Thus, it is possible to prevent the former disadvantage, but is impossible to obtain the latter advantage.

Therefore, by prompting the user to predict a change in measurement value, it becomes possible to give the user an opportunity to look back living activities of the day, thereby allowing the user to maintain interest in body weight and improvement in his/her lifestyle. The previous measurement value for comparison may be any previous measurement as long as it facilitates the user's prediction about a change in body weight. In this embodiment, the previous measurement value is set as an average of measurement values measured in the preceding week. Alternatively, the previous measurement value may be an average of all measurement values measured in the period from a measurement starting day to the preceding week, or may be an average of a given number of measurement values, e.g., 2 or 3 measurement values, measured in the past, or may be a measurement value of the previous day. However, the system 100 according to this embodiment can be implemented without input of the prediction. Further, the prediction is not limited to increase/decrease, but may include “no change”.

When the smartphone 102 receives the tag selection and the prediction input through the input part 503 (S612), the control part 501 of the user-side device 102 operates to compare the measurement value which is targeted for the prediction input, with a previous measurement value, thereby performing a prediction correct/incorrect determination (S613). Here, the previous measurement value is set as an average of measurement values measured in the preceding week (August 09 to August 15), specifically 81.2 kg. For example, assuming that the measurement value which is targeted for the prediction input is 80.3 kg, this measurement value decreases as compared with 81.2 kg as the previous measurement value. Thus, when the prediction input is “decrease”, it is determined to be correct. On the other hand, when the reduction input is “increase”, it is determined to be incorrect. In this embodiment, the average of the previous measurement values is preliminarily calculated and stored in the storage part 504 of the user-side device 102, and read out during the correct/incorrect determination to execute the comparison processing. Alternatively, the average may be stored in the storage part 404 of the body weight scale 101, or may be sequentially calculated based on given previous values.

Upon completion of the prediction correct/incorrect determination (S613), the control part 501 operates to store, in the storage part 504, the selected tag and a result of the correct/incorrect determination, together with the measurement value of body weight and the measurement time each transmitted from the body weight scale 101, in association with this measurement value and time as a measurement data (S614). In this embodiment, the measurement data has a data structure as shown in Table 1.

TABLE 1

| Measurement | Measurement | | Prediction | Correct/Incorrect |
|---|---|---|---|---|
| Time | value (kg) | Tag | Input | of Prediction |
| August 09, 7:50 | 81.5 | 0 | — | — |
| August 10, 7:30 | 81.2 | 0 | — | — |
| August 11, 9:00 | 81.6 | 1 | — | — |
| August 12, 19:00 | 81.4 | 1 | — | — |
| August 13, 21:00 | 82.5 | 4 | — | — |
| August 14, 7:52 | 81.2 | 0 | — | — |
| August 15, 16:00 | 79.4 | 2, 3 | — | — |
| August 16, 7:54 | 80.3 | 0, 3 | 0 | 1 |
| August 17, 15:00 | 79.8 | 3 | 0 | 1 |
| August 18, 7:45 | 80.7 | 0 | 1 | 0 |
| August 19, 7:30 | 80.8 | 0 | 0 | 1 |
| August 20, 22:00 | 82 | 4 | 0 | 0 |
| August 21, 7:38 | 80.4 | 2 | 1 | 0 |
| August 22, 7:20 | 81.5 | 0, 5 | 0 | 0 |
| August 23, 7:50 | 81 | 0, 5 | 0 | 1 |
| August 24, 21:00 | 81.2 | 2, 5 | 0 | 0 |

In Table 1, the measurement time is a date-and-time when the user measured his/her body weight by the body weight scale, and the measurement value is the measured body weight. The tag is a tag number corresponding to a tag selected by the user. Here, the state after urination at the time of awakening is denoted by 0, and the state in which there is an influence of meal is denoted by 1. The state after taking a bath is denoted by 2, and the state after exercise is denoted by 3. Further, the dressed state is denoted by 4, and the state during a menstrual period is denoted by 5. The prediction input is a number corresponding to the body weight increase/decrease prediction input by the user. Here, a prediction of decrease is denoted by 0, and a prediction of increase is denoted by 1. The correct/incorrect of prediction indicates a result of determination made in the prediction correct/incorrect determination (S613), based on the prediction input by the user, wherein the incorrect prediction is denoted by 0, and the correct prediction is denoted by 1. For example, in Table 1, on August 22, the measurement was performed at 7:20, and the measurement value was 81.5 kg. Further, the selected tag was 0 (state after urination at the time of awakening), and 5 (state during a menstrual period). Although the prediction was 0 (decrease), it was 0 (incorrect). In this example, the user started the measurement from August 09. Thus, until August 15, there is not any measurement value of the preceding week necessary for the prediction input, so that the prediction input and the prediction correct/incorrect determination are not performed until August 15.

After storing the measurement data in the storage part 504 of the smartphone 102 (S614) or receiving no measurement completion notice in a standby mode to wait for the measurement completion notice (S610), the control part 501 operates to wait for a measurement result display request (S620). In this embodiment, the measurement result display request is issued when the user performs an input manipulation for displaying a measurement result, through the input part 503 of the smartphone 102. When no measurement result display request has been issued, the control part 501 operates to return to the standby mode to wait for the measurement completion notice (S610).

When the measurement result display request has been issued, the control part 501 operates to perform processing of determining a set of measurement data satisfying the set presentation timing condition (S621). In this embodiment, the presentation timing condition is set as date-of-week, as mentioned above. Here, the description will be made based on an example where the presentation timing condition is set to Sunday at 12:00 a.m. Each of a plurality of measurement data includes a measurement value of body weight, a measurement time, and a tag and a correct/incorrect determination result each associated with the measurement value. Thus, based on the measurement time included in each of the measurement data, the control part 501 operates to determine whether or not each of the measurement data satisfies the presentation timing condition. More specifically, comparing Sunday at 12:00 a.m. in a week next to a week including the measurement time of a certain measurement data, with a current date-and-time, when the current date-and-time is on or after Sunday at 12:00 a.m. in the next week, the certain measurement data is determined to satisfy the presentation timing condition. In the case where the presentation timing condition is set as the elapse of the required time period, when a value obtained by subtracting the measurement time of a certain measurement data from the current date-and-time is equal to or greater than the required time period, the certain measurement data is determined to satisfy the presentation timing condition.

The control part 501 operates to display a measurement result based on a set of measurement data determined to satisfy the presentation timing condition, on the display part 502 of the smartphone 102 (S622). On the other hand, the control part 501 operates to avoid displaying any measurement result based in the measurement data determined to fail to satisfy the presentation timing condition. A measurement result is based on each of the measurement data, and may be a measurement value itself therein or may include statistical data such as an average based on measurement values. After displaying the measurement result, the control part 501 of the user-side device 102 operates to return to the standby mode to wait for the measurement completion notice.

As one example, a case will be described in which the presentation timing condition was set to Sunday at 12:00 a.m., wherein the measurement was started on August 09 (Sun) and performed everyday until August 24 (Mon), and the measurement result presentation request was issued on August 24 at 15:00. In this case, the control part 501 operates to determine whether or not August 24 at 15:00, which is a request date-and-time when the measurement result presentation request was issued, satisfies the presentation timing condition, with respect to each measurement data. Thus, with regard to measurement data in the period from August 09 (Sun) to August 22 (Sat), the request date-and-time is beyond Sunday of a week next to a week including the measurement time of each of the measurement data, i.e., August 16 or 23 at 12:00 a.m. Thus, a measurement result regarding these measurement data is presented. On the other hand, with regard to measurement data on August 23 and August 24, the request date-and-time is not beyond August 30 at 12:00 a.m., which is Sunday of a week next to a week including the measurement time of each of the measurement data. Thus, these measurement data do not satisfy the presentation timing condition, so that a measurement result based on these measurement data is not presented.

As shown in FIG. 7(b), as the latest body weight, 80.8 kg which is an average of measurement values in the preceding week from August 16 to August 22 is displayed and further—0.4 kg which is a difference between this average and an average of measurement values before the preceding week is displayed. Further, 81.0 kg which is an average of all measurement values in the period from the measurement starting day to the preceding week is displayed. Further, a measurement result is presented by displaying the measurement values in the form of a transition graph. The average to be graphically displayed may be moving average. As shown in FIG. 7(b), the graph as the measurement result based on the measurement data in the period from August 09 (Sun) to August 22 (Sat) satisfying the presentation timing condition is displayed, whereas the measurement data on August 23 and August 24 are not displayed because they fail to satisfy the presentation timing condition.

As mentioned above, the body weight of the user varies depending on the state of the user at the time of the measurement. Therefore, a judgment about the progress of body weight reduction cannot be adequately made even by focusing on only individual measurement values. By presenting, as a measurement result, an average of measurement data after a certain amount of time has elapsed, instead of presenting a measurement result of each day immediately after the measurement, and presenting a transition of measurement values in the form of a graph, as in this embodiment, it becomes possible to dispassionately figure out the tendency of changes in body weight without excessively focusing on only individual measurement values. Further, as information to be presented, only an average or moving average, or a transition, of past measurement data, may be presented, without presenting any individual measurement data. By avoiding presenting individual data indicative of no significant change in body weight, it becomes possible to prevent the user from reacting nervously to insignificant changes in body weight.

Further, as shown in FIG. 7(b), each plot of the graph may be indicated together with the tag associated with the plotted measurement value in a visually recognizable manner. In a case where each measurement value is associated with a plurality of tags, the tags may be prioritized so as to present only a highest-priority one of the tags, or all the tags may be displayed in characters or different colors, in a visually recognizable manner.

In the case where a measurement result of each day is presented immediately after the measurement, when there is a change in measurement value as compared with that of the preceding day, the user can think by which of activities of the day the change is caused. However, in this embodiment, the user can look at a measurement result of the day only on or after next Sunday. Thus, when looking at the measurement result, it is difficult for the user to remember activities of the day when an individual measurement value of body weight was measured, and think by which of the activities a change in measurement value is caused. Therefore, in this embodiment, the tag is used. The tag is indicative of a factor related to the health-related information of the user at the time of the measurement of the health-related information of the user. Thus, the tag can help the user to remember activities of the day when an individual measurement value was measured, and allows the user to easily figure out by what kind of the user's activities and in what kind of pattern a change in measurement value is caused.

Further, as shown in FIG. 7(b), statistical information such as an average body weight with respect to each tag may be presented. This allows the user to understand how each of the factors related to the health-related information exerts an influence on the health-related information. For example, from the statistical information, it is possible to understand that the body weight becomes relatively low after taking a bath or after exercise, whereas the body weight becomes relatively high when there is an influence of meal or during a menstrual period. Thus, although the user is apt to understand that the reduction in body weight succeeded when he/she looks at only the body weight after taking a bath or after exercise, the above statistical information makes it possible to correct such an understanding, i.e., to show that it is incorrect to understand a relatively low body weight measured after taking a bath or after exercise, as a basis for the success of the reduction in body weight.

Further, results of the prediction correct/incorrect determination may be presented as the measurement result. In this embodiment, as shown in FIG. 7(c), a correct prediction rate and a body weight prediction graph are presented. In the example illustrated in FIG. 7(c), it is presented that only three predictions are correct among seven predictions, and the correct prediction rate is 42%. Further, in the body weight prediction graph, because no prediction input was made in the period from August 09 to August 15, no result of the prediction correct/incorrect determination in this period is indicated. On August 16, 17 and 19, the prediction was decrease, and correct. On August 18 and 21, the prediction was increase and incorrect, and, on August 20 and 22, the prediction was decrease and incorrect. Because the presentation timing condition is not satisfied on and after August 23, no result of the prediction correct/incorrect determination in this period is indicated. Whether each of the results of the prediction correct/incorrect determination in the period from August 16 to August 22 is a prediction of increase which is correct, a prediction of decrease which is correct, a prediction of increase which is incorrect, or a prediction of decrease which is incorrect, is displayed in the form of a graph. On the above graph, a line of 81.2 kg as an average body weight in the period from August 09 to August 15 is displayed as a reference line. This makes it possible to inform the user of information about how close the value of body weight determined by user's feeling conforms to an actual measurement value. A high rate of incorrect prediction means that user's understanding of body weight is wrong.

For example, if the user erroneously understands that the body weight immediately decreases only by performing mild exercise once, he/she will make a prediction that the body weight has decreased after performing mild exercise. However, normally, the body weight never decreases immediately after performing mild exercise once, so that a prediction of decrease becomes incorrect. Thus, the body weight prediction graph allows the user to understand a lack of physical exercise, and makes it possible to prompt the user to improve his/her lifestyle, such as an increase in the amount of physical exercise or a decrease in the amount of calorie intake, in daily life. Further, if the user who is unaware that the body weight increases during a menstrual period makes a prediction of decrease, irrespective of during a menstrual period, this prediction becomes incorrect. Because of presentation of the incorrect prediction, the user becomes aware of the fact that her understanding is wrong, and understands that the body weight is apt to increase during a menstrual period. Thus, the increased measurement value does not cause the user to be unnecessarily depressed

Further, as shown in FIG. 7(d), the measurement result may be displayed in the form of a table. In this embodiment, the measurement result includes measurement times, measurement values, tags, prediction inputs, and results of the prediction correct/incorrect determination.

As mentioned above, in the first embodiment, only a measurement result based on the measurement data satisfying the presentation timing condition is presented to the user, but measurement result based on the measurement data failing to satisfy the presentation timing condition is not presented to the user. Here, a measurement result is updated once per week, and measurement results measured in the week cannot be checked until next updating. If a measurement value of body weight is presented to a user immediately after the measurement as in a conventional body weight scale, the use is likely to react nervously to the measurement value, and to be discouraged when the measurement value is not as good as the user expected to be, thereby stopping the measurement of body weight. However, in the first embodiment, such a measurement value is not presented until Sunday in the next week. Therefore, when the user looks at a measurement result, a certain amount of time has elapsed from the time of the measurement. This makes it possible to diminish user's excessive interest in each measurement value, and allow the user to objectively understand a long-term transition of measurement value.

A measurement value of body weight or the like is likely to vary depending on activities of a day. If the user can look at a measurement value at the time of measurement, he/she can understand the reason for the variation, e.g., a high measurement value was caused by a large size of a meal of the day. However, in the first embodiment, a certain amount of time has elapsed from the time of the measurement to presentation. Thus, it is difficult for the user to remember with what kind of a factor related to body weight (body weight-related factor) existed at the time of the measurement. Therefore, by assigning, to a measurement value, a tag serving as an index indicative of a body weight-related factor at the time of the measurement, it becomes possible to allow the user to easily understand the reason for a change in body weight even when a measurement result based on the measurement value is presented afterward. Further, the user can look at a transition of measurement values assigned with the same tag. This allows the user to more easily understand an influence of a body weight-related factor indicated by each tag, on body weight.

In the case where a measurement value can be checked immediately after the measurement, when the measurement value becomes worse, the user is likely to be discouraged, or is likely to try to improve his/her habitus in a more carefully controlled manner. However, in the invention, a measurement result cannot be checked immediately after the measurement, so that there is a possibility that user's motivation for improvement in daily habitus is diminished. Therefore, in the first embodiment, the user is prompted to make a prediction about increase/decrease in measurement value, during the measurement. This makes it possible to give the user an opportunity to look back and reflect on living activities of each day, and motivate the user to improve his/her habitus. Further, by presenting correct/incorrect of the prediction in a measurement result, it becomes possible for the user to become aware of whether or not user's understanding about a relationship between his/her living activity and body weight is right, and, if wrong, to correct the understanding.

Any measurement data determined to satisfy the presentation timing condition is flagged and stored as a part of the plurality of measurement data in the storage part 504. Then, when the measurement result display request is issued, a measurement data may be presented based on the flagged measurement data which are deemed as the measurement data satisfying the presentation timing condition. This can facilitate processing for a determination as to whether or not each measurement data satisfies the presentation timing condition, to be performed every time a measurement result is displayed. The determination as to whether or not each measurement data satisfies the presentation timing condition, based on a measurement time, may be performed for unflagged measurement data, or may be periodically performed at given time intervals.

In the embodiment, a measurement time (clock time) is used as information indicative of the measurement timing, and whether or not each measurement data satisfies the presentation timing condition is determined based on the measurement time and the timing to present the measurement result. Alternatively, it may be determined based on the number of measurements performed in the period after the measurement through until the present timing. For example, the smartphone 102 may be configured to, when seven or more measurements are performed in the period after the measurement through until the present timing, present a measurement result based on a measurement data of the measurement. In one example, each measurement data may include a measurement number-of-times value to be incremented by one every time a new measurement is performed after a measurement value included in the measurement data is measured, wherein a measurement result based on measurement data each having a measurement number-of-times value of 7 or more at the present timing can be displayed. The measurement number-of-times value indicates that a measurement data including the measurement number-of-times value was measured at an n-th latest timing (n corresponds to the measurement number-of-times value) on the basis of the present timing, so that it is indicative of the measurement timing. Alternatively, each measurement data may include a measurement number indicative of the order of measurement, as the information indicative of the measurement timing, and a value obtained by subtracting 7 from a measurement number of a lastly-measured measurement data at the present timing may be defined as a presentable maximum measurement number, wherein measurement results based on measurement data each having a measurement number less than the presentable maximum measurement number is allowed to be presented. A measurement number-of-times value of a certain measurement data can be obtained by comparing a measurement number of the certain measurement data with a measurement number of a lastly-measured measurement data at the present timing, so that the measurement number indicative of the order of measurement is information indicative of the number of measurements performed after the measurement of a measurement value included in the certain measurement data. As long as the user performs the measurement in each day, he/she can look at a measurement result only after the elapse of one week since the measurement. Thus, it is possible to obtain the same advantageous effects as those in the configuration in which whether or not each measurement data satisfies the presentation timing condition is determined based on the measurement time and the present timing.

The presentation timing condition based on a measurement time and the presentation timing condition based on a measurement number-of-times value may be combined together. For example, such a presentation timing condition may be set to allow a measurement result based on a certain measurement data to be presented only after one week has elapsed since the measurement, and seven or more measurements have been subsequently performed.

Further, when it can be determined, based on a criterion based on a value indicative of a tendency of past measurement values, that a measurement value does not largely deviate from the tendency of the past measurement values, this measurement value may be determined to satisfy the presentation timing condition. For example, when a difference between an average of all measurement values stored in the storage part 504 and a measurement value which is targeted for the determination falls within the range of +5% to −5%, the target measurement value is determined to satisfy the presentation timing condition. On the other hand, when the difference is out of the above range, the target measurement value is determined not to be presented. In the case where the presentation timing condition is based on the tendency of past measurement values, it becomes free of being bound by a time period or the number of measurements after the measurement, so that it is possible to present an effect of improved living activities as soon as possible without unnecessarily keep the user waiting for the presentation. This makes it possible to prevent the user from giving up the improvement in living activities due to a suspicion that a sufficient effect cannot be obtained, even though the effect of improved living activities is actually obtained (avoidance of false-negative).

The criterion based on a value indicative of the tendency of past measurement values may be based on how much difference there is with respect to: an average of all measurement values in the period from a measurement starting day to the preceding week; a moving average of measurement values corresponding to a given number of past measurements such as past 2 or 3 measurements; or an average of transitional minimum ones of the measurement values. In addition to the above, any criterion may be used which allows the determination as to whether or not a measurement value largely deviates from the tendency of past measurement values. In a case where a measurement result to be presented is an average, whether or not an average including a target measurement value largely deviates from the tendency of past measurement values may be determined.

Further, the presentation timing condition based on the transition of past measurement values may be combined with the presentation timing condition based on a measurement time and/or a measurement number-of-times value. For example, when a difference between an average of all measurement values stored in the storage part 504 and a target measurement value falls within the range of +5% to −5%, a measurement result based on the target measurement value is presented even if the target measurement value does not satisfy the presentation timing condition based on a measurement time and/or a measurement number-of-times value. On the other hand, when the difference is out of the above range, the measurement result is not presented until the target measurement value satisfies the presentation timing condition based on a measurement time and/or a measurement number-of-times value.

In the first embodiment, the functions of the setting of the presentation timing condition, the storing of measurement data, the prediction correct/incorrect determination, and the determination based on the presentation timing condition are executed by the smartphone 102. Alternatively, the body weight scale 101 may be configured to execute a part of the functions.

In the first embodiment, only the body weight is measured as the health-related information. Alternatively, instead of or in addition to the body weight, a biological impedance for measuring a body fat may be measured. As the health-related information, another biological information such as blood pressure, heart rate, or blood-sugar level, and physical activity-related information such as the number of steps can also be applied and handled by similar information processing. The health-related information measurement device 101 may be constructed as a wearable device for measuring the health management information such as the number of steps or blood pressure. A measurement value may be a maximum or minimum value among values measured for a given time period, e.g., one day, or an average of the measured values.

In a case where a pedometer is used as the health-related information measurement device, the pedometer counts up the number of steps every time the user walks. Then, a total number of steps for a given time period, e.g., from 00:00 in the morning to 12:00 at night, is used as a measurement value on a day-to-day basis. In this case, a measurement timing may be set to 12:00 at night which id an ending time of the given time period. The given time period may be a part of one day, or may be several days. Alternatively, the given time period may be started at a timing when the pedometer accepts a start instruction input from the user, and ended at a timing when the pedometer subsequently accepts a termination instruction input from the user. Although it is important for health promotion or the like to continuously walk every day by a given number or more of steps, a user unnecessarily gets satisfaction from a measurement value measured by the pedometer on the day when he/she occasionally walked by a relatively large number of steps, and thereby will neglect walking, from the next day, in some cases. On the other hand, there are also many cases where a user is largely discouraged by a small number of steps measured by the pedometer on the day when he/she occasionally walked by a relatively small number of steps, and stops the measurement after that, finally leading to abandonment of an effort to improve his/her health state. The system 100 according to the first embodiment makes it possible to allow the user to continuously perform living activities for health promotion or the like, without reacting nervously to a measurement value of each day.

### Second Embodiment

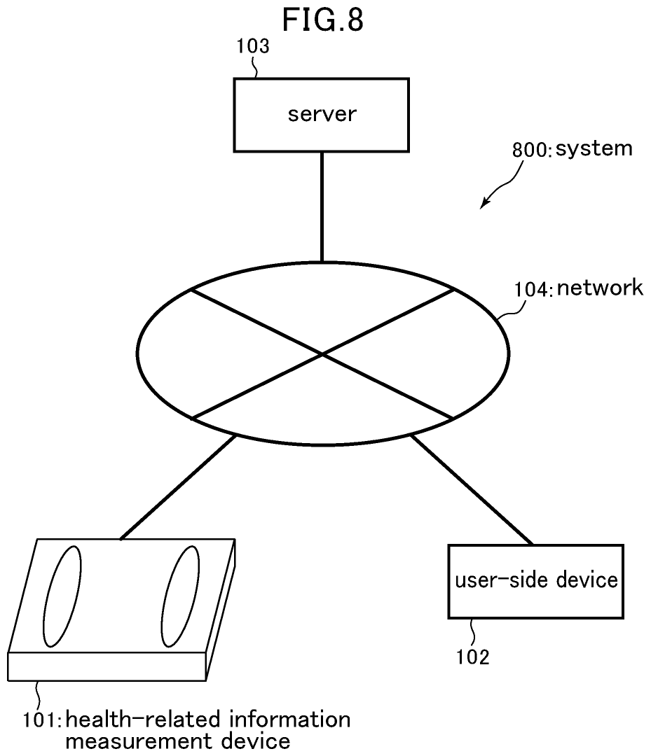

As shown in FIG. 8, a health-related information management system according to a second embodiment of the present invention is different from the first embodiment in that, in addition to a health-related information measurement device 101 and a user-side device 102, the system comprises a server 103, wherein they are connected to each other via a network 104. The following description will be made with a focus on a difference from the first embodiment.

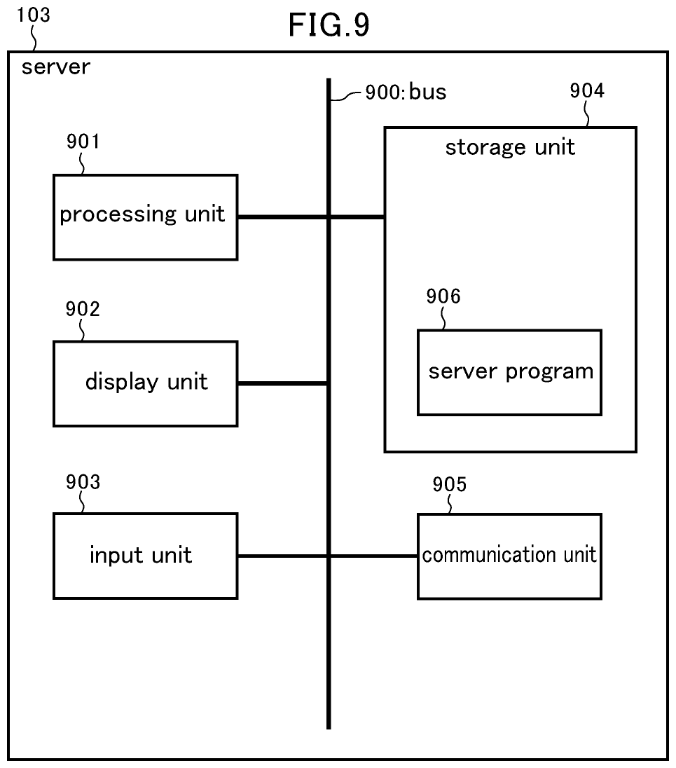

FIG. 9 is a hardware configuration diagram of the server 103 as one electronic device in the system according to the second embodiment. The server 103 is an electronic device which comprises a processing unit 901, a display unit 902, an input unit 903, a storage unit 904, and a communication unit 905. These units are connected to each other via a bus 900. However, the system may be configured such that two or more of the units are connected together, on an as-needed or case-by-case basis. The display unit 902 has a function of displaying information for a user. The input unit 903 has a function of accepting an input from the user, and is composed of a keyboard, a mouse or the like. The storage unit 904 stores therein a server-operating program 906. The storage unit 904 may be any type of storage unit such as a non-volatile memory or a volatile memory, as long as it is capable of storing information therein. The communication unit 905 that performs wire communication using an Ethernet (trademark) cable or the like, or wireless communication using mobile communication, wireless LAN or the like, to establish connection to the network 104.

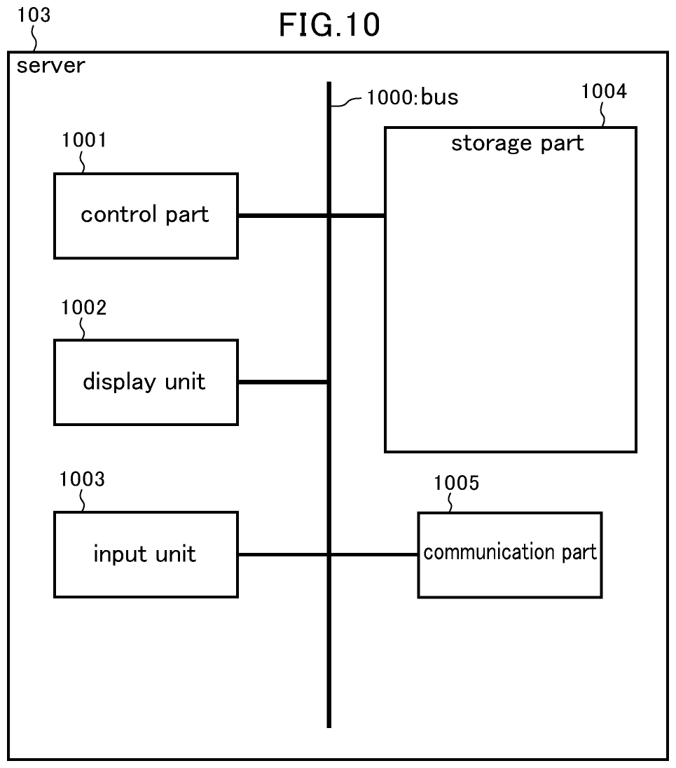

FIG. 10 is a functional block diagram of the server 103 in the system according to the second embodiment. The server 103 comprises a control part 1001, a display part 1002, an input part 1003, a storage part 1004, and a communication part 1005. The control part has a function of executing controls such as information processing. The display part 1002 has a function of allowing the user to visually view information. The input part 1003 has a function of accepting an input from the user. The storage part 1004 has a function of storing therein data and others. The communication part 1005 has a function of performing transmitting and receiving of information with respect to another device. In the second embodiment, the above functions are realized by executing the program 906 in the processing unit 901 illustrated in FIG. 9 to cause the hardware to co-operate with one another. Alternatively, these functions may be realized based on hardware by providing one or more electronic circuits or the like capable of realizing the functions. Further, a plurality of the servers 103 may be provided and configured to achieve these functions in cooperation with each other.

The health-related information measurement device 101 and the user-side device 102 are implemented by the functional blocks illustrated in FIGS. 4 and 5 realized by the same hardware configurations as those in the first embodiment illustrated in FIGS. 2 and 3. Here, the health-related information measurement device 101 and the user-side device 102 are connected to the network 104 via a wire or wireless LAN, instead of a Bluetooth (trademark) connection, and are connected to each other through the server 104.

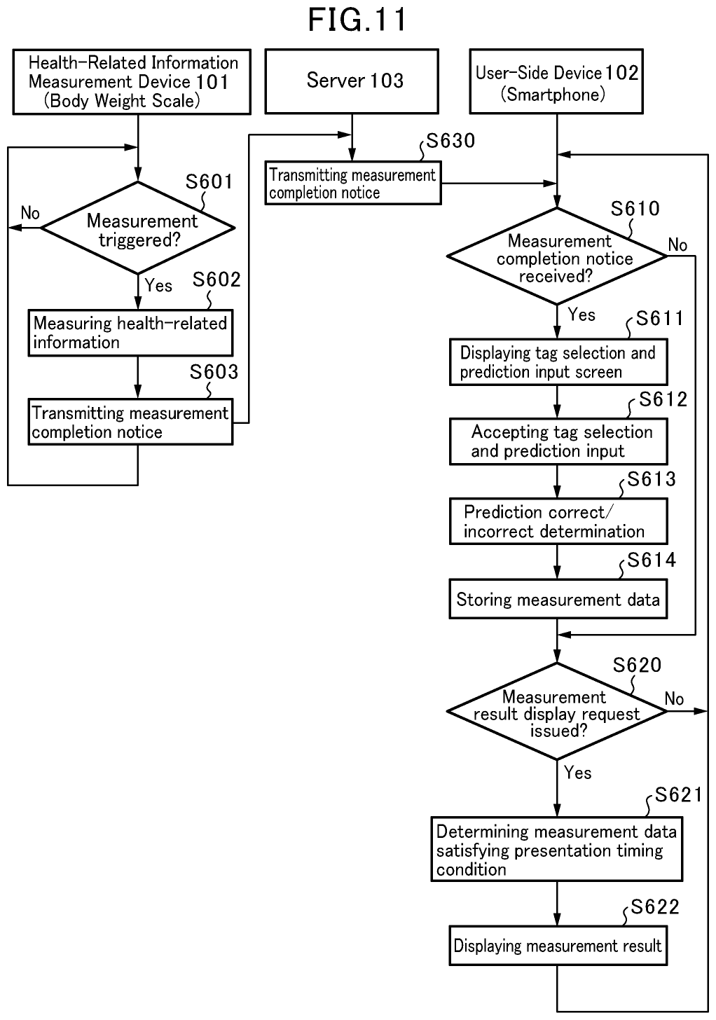

A flowchart of a body weight measurement operation as a health-related information measurement operation by the body weight scale 101 and an information processing operation by the smartphone 102 is shown in FIG. 11. In the second embodiment, for the information processing, the health-related information measurement device 101 and the user-side device 102 are connected to each other through the server 103, instead of being directly connected to each other via a Bluetooth (trademark) connection. Thus, a measurement completion notice is transmitted from the body weight scale 101 to the server 103. Then, in response to receiving the measurement completion notice, the server 103 further transmits the measurement completion notice to the user-side device 102 (S630), and the user-side device 102 receives the measurement completion notice. The remaining respects are the same as those in the first embodiment.

### Third Embodiment

As shown in FIG. 8, in a health-related information management system 800 according to a third embodiment of the present invention, a health-related information measurement device 101 and a user-side device 102 are connected to each other via a network 104 and a server 103 as with the second embodiment. However, the third embodiment is different from the second embodiment in terms of information processing for presenting a measurement result. The following description will be made with a focus on a difference from the second embodiment.

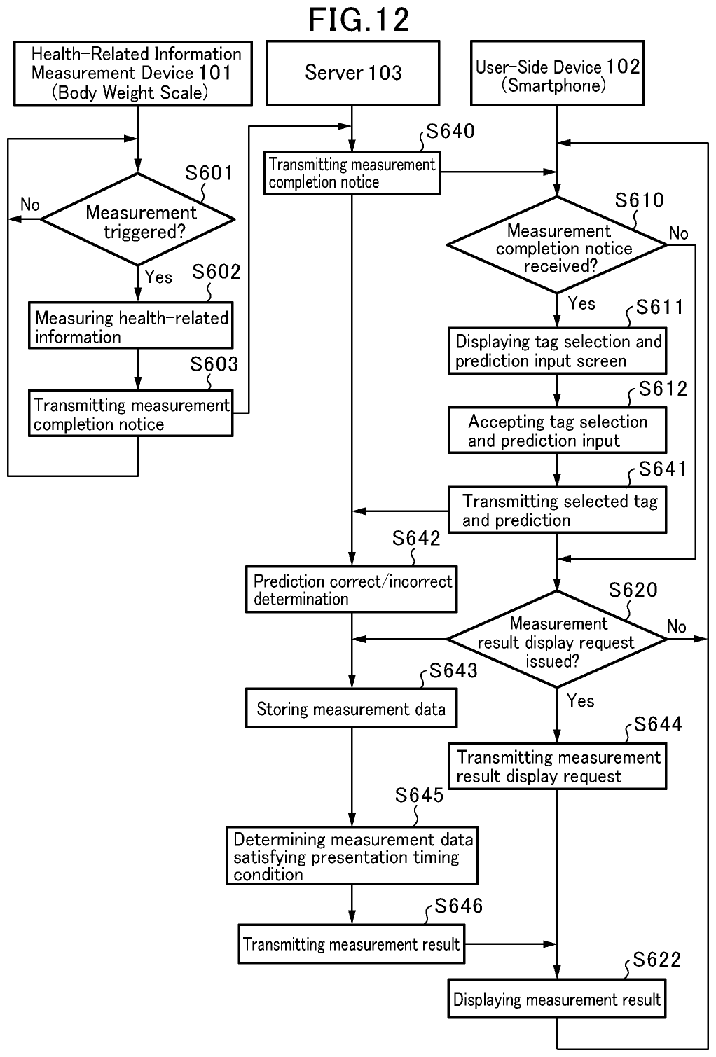

A flow of information processing for presenting a measurement result in the third embodiment is shown in FIG. 12. In the third embodiment, a measurement completion notice to be transmitted from the body weight scale 101 contains a measurement value and a measurement time as with the second embodiment, whereas a measurement completion notice to be transmitted from the server 103 does not contain the measurement value and the measurement time. Specifically, the measurement completion notice to be transmitted from the server 103 is a notice for simply instructing the user-side device 102 to display a tag input screen for prompting a user to tag a completed measurement. In response to receiving a measurement completion notice containing a measurement value transmitted from the body weight scale 101, the server 103 transmits, to the smartphone 102, a measurement completion notice for instructing the smartphone 102 to display the tag input screen (S640). Then, as with the second embodiment, in response to receiving the measurement completion notice, the smartphone 102 displays a tag selection and prediction input screen (S611), and accepts a tag selection and a prediction input (S612). Subsequently, in the third embodiment, the smartphone 102 transmits, to the server 103, a selected tag and a prediction input by the user (S641). In response to receiving the selected tag and the prediction, the server 103 performs prediction correct/incorrect determination proceeding in the same manner as the prediction correct/incorrect determination proceeding to be executed by the user-side device 102 in the first and second embodiments (S642), and stores, in the storage part 1004 of the server 103, prediction correct/incorrect information determined through the prediction correct/incorrect determination proceeding, together with the measurement value and the measurement time transmitted from the body weight scale 101 and the selected tag and the prediction transmitted from the user-side device 102, as measurement data.

The user-side device 102 waits for an input of a measurement result display request by the user (S620), and, upon accepting the measurement result display request, transmits the measurement result display request to the server 103 (S644). In response to receiving the measurement result display request, the server 103 determines, in measurement data stored in the storage part 1004, measurement data satisfying the presentation timing condition (S645), and transmits, to the user-side device 102, a measurement result based on the measurement data satisfying the presentation timing condition (S646). This determination and measurement result forming processing is the same as the processing to be executed by the user-side device 102 in the first and second embodiments. The user-side device 102 displays the received measurement result on the display part (S622).

In the third embodiment, the measurement result forming processing may be executed by the user-side device 102.

### Fourth Embodiment

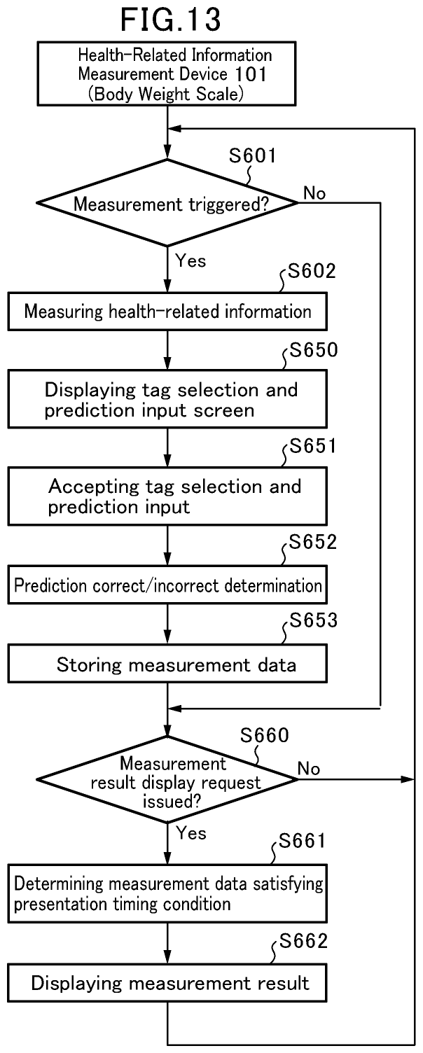

A fourth embodiment of the present invention is different from the first to third embodiments in that a body weight scale as a health-related information measurement device 101 executes the entire measurement data presentation processing, as shown in FIG. 13. In the fourth embodiment, the body weight scale 101 comprises, as hardware, a display unit and an input unit, which realize functional parts: a display part and an input part. When a user steps on the body weight scale, the body weight scale starts a measurement of body weight (S601) to measure the body weight of the user (S602). Upon completion of the measurement of body weight, the display part of body weight scale operates to display a measurement time, and display a tag selection and prediction input screen (S650). In response to accepting a selected tag and a prediction from the user through the input part of the body weight scale (S651), the body weight scale 101 performs a prediction correct/incorrect determination (S652), and stores, in the storage part 504 of the body weight scale 101, the measurement time, a measurement value, the selected tag, the prediction, and a result of the prediction correct/incorrect determination, as a measurement data (S653). Subsequently, upon input of a measurement result display request through the input part of the body weight scale 101, the body weight scale 101 selects, from measurement data stored in the storage part 504, measurement data satisfying the presentation timing condition (S661), and displays, on the display part of the body weight scale 101, a measurement result based on the selected measurement data (S662).

Although the present invention has been described by way of the above embodiments in order to express the present invention, it should be noted that the present invention is not limited to such embodiments in any way. It is obvious to a person of ordinary skill in the art that the present invention may be implemented in various forms or configurations without departing from the spirit and scope thereof as set forth in appended claims.

### LIST OF REFERENCE SIGNS

100: system
101: health-related information measurement device
102: user-side device
103: server
104: network
200: bus
201: processing unit
202: display unit
203: input unit
204: storage unit
205: communication unit
206: health-related information measurement device program
207: measurement unit
300: bus
301: processing unit
302: display unit
303: input unit
304: storage unit
305: communication unit
306: user-side device program
401: control part
404: storage part
405: communication part
407: health-related information measurement part
501: control part
502: display part
503: input part
504: storage part
505: communication part
800: system
900: bus
901: processing unit
902: display unit
903: input unit
904: storage unit
905: communication unit
906: server-operating program
1001: control part
1002: display part
1003: input part
1004: storage part
1005: communication part

1.-15. (canceled)

16. A health-related information management system comprising:
one or more electronic devices, wherein
at least one of the one or more electronic devices:
measures measurement data including a measurement value and a measurement timing of the measurement value;
stores the measurement data and the measurement timing;
determines, using the measurement timing and a timing to present a measurement result using the measurement data, whether the measurement data satisfies a pre-set presentation timing condition; and
presents, as the measurement result, only measurement data among the measurement data that satisfy the pre-set presentation timing condition.

17. The health-related information management system of claim 16, wherein
the measurement timing includes a measurement time of the measurement value, and
the pre-set presentation timing condition includes:
a predetermined day-of-week or day-of-month after the measurement time, or
a timing after a given time period has elapsed from the measurement time.

18. The health-related information management system of claim 16, wherein
the measurement timing includes information reflecting a number of measurements performed after a first measurement of the measurement value, and
the pre-set presentation timing condition includes a timing after a predetermined number of measurements have been performed since the first measurement of the measurement value.

19. The health-related information management system of claim 16, wherein the measurement value of the measurement data satisfies the pre-set presentation timing condition when the measurement value satisfies a criterion based on a value indicative of a tendency of past measurement values.

20. The health-related information management system of claim 16, wherein
at least one of the one or more electronic devices further accepts an index input,
the index input is associated with the measurement value and indicates a factor related to a health-related information of a user when the health-related information of the user is measured, and
the measurement data includes the index input.

21. The health-related information management system of claim 20, wherein
the health-related information of the user includes a body weight of the user, and
the index input indicates at least one of a state after urination at a time of awakening, a state after eating, a state after taking a bath, a state after exercise, a dressed state, and a state during a menstrual period.

22. The health-related information management system of claim 16, wherein the one or more electronic devices comprise a health-related information measurement device and a user-side device.

23. The health-related information management system of claim 16, wherein at least one of the one or more electronic devices further:
receives a prediction input that predicts whether the measurement value increased or decreased from a previous measurement value; and
compares the measurement value with the previous measurement value to determine whether the prediction input is correct, wherein
the measurement data includes a result of the determination.

24. The health-related information management system of claim 16, wherein a graph based on the measurement data, including measurement values measured within a given time period, is displayed as the measurement result.

25. The health-related information management system of claim 16, wherein the one or more electronic devices comprise: a health-related information measurement device; a user-side device; and a server connected to the health-related information measurement device and the user-side device via a network.

26. A user-side device of a health-related information management system, the user-side device comprising:
a processor that presents only measurement data, among measurement data measured by a health-related information measurement device of the health-related information management system, that satisfy a pre-set presentation timing condition as a measurement result, wherein
the measurement data includes measurement values and measurement timing of the measurement values, and
the measurement data is determined to satisfy the pre-set presentation timing condition based on the measurement timing and a timing to present the measurement result using the measurement data.

27. A health-related information management method comprising:
measuring measurement data including a measurement value and a measurement timing of the measurement value;
storing the measurement data and the measurement timing;
determining, using the measurement timing and a timing to present a measurement result using the measurement data, whether the measurement data satisfies a pre-set presentation timing condition; and
presenting, as the measurement result, only measurement data among the measurement data that satisfy the pre-set presentation timing condition.

28. A non-transitory computer readable medium (CRM) storing computer readable program code for one or more electronic devices of a health-related information management system embodied therein that:
measures measurement data including a measurement value and a measurement timing of the measurement value;
stores the measurement data and the measurement timing;
determines, using the measurement timing and a timing to present a measurement result using the measurement data, whether the measurement data satisfies a pre-set presentation timing condition; and
presents, as the measurement result, only measurement data among the measurement data that satisfy the pre-set presentation timing condition as the measurement result.

29. A health-related information management method performed by a user-side device in a health-related information management system, the method comprising:
presenting only measurement data, among measurement data measured by a health-related information measurement device of the health-related information management system, that satisfy a pre-set presentation timing condition as a measurement result, wherein
the measurement data includes measurement values and measurement timing of the measurement values, and
the measurement data is determined to satisfy the pre-set presentation timing condition based on the measurement timing and a timing to present the measurement result using the measurement data.

30. A non-transitory computer readable medium (CRM) storing computer readable program code for a user-side device of a health-related information management system embodied therein that:
presents only measurement data, among measurement data measured by a health-related information measurement device of the health-related information management system, that satisfy a pre-set presentation timing condition as a measurement result, wherein
the measurement data includes measurement values and measurement timing of the measurement values, and
the measurement data is determined to satisfy the pre-set presentation timing condition based on the measurement timing and a timing to present the measurement result using the measurement data.
